(19) United States
(12) Patent Application Publication
(10) Pub. No.: US 2021/0260399 A1
(43) Pub. Date: Aug. 26, 2021

(54) System of a Wearable Laser Device and an AI or ML Based Platform with Smart Virtual Assistant for Monitoring and Treatment of Pain including Peripheral Neuropathy

(71) Applicant: Abijith Kariguddaiah, Danville, CA (US)

(72) Inventor: Abijith Kariguddaiah, Danville, CA (US)

(21) Appl. No.: 17/318,171

(22) Filed: May 12, 2021

Related U.S. Application Data
(63) Continuation-in-part of application No. 15/488,339, filed on Apr. 14, 2017.

(51) Int. Cl. A61N 5/06; G16H 10/20; G16H 40/40

(52) U.S. Cl. CPC A61N 5/0613; A61N 2005/067; G16H 40/40; G16H 10/20

(57) ABSTRACT

A system of a wearable laser device and an AI or ML based Platform with “smart virtual assistant” is provided for monitoring and treatment of pain including from Peripheral Neuropathy. The system is made of a plurality of wearable devices with an embedded laser diodes of a low frequency/intensity for treatment of pain and electronic unit within each of the plurality of wearable device. The electronic unit includes a plurality of various sensors to monitor blood glucose level, flexibility of movement and other vital body data of the user, a control unit, and a communication unit to transmit and store the sensory data over the central cloud server. The system further includes a computer implemented platform with an AI powered smart virtual assistant that processes sensory data in real time and notifies the user, personal physician and all the persons of concern, if any medical emergency arises.

### CROSS REFERENCE TO RELATED APPLICATIONS

The present application claims priority to and benefit from U.S. patent application Ser. No. 15/488,339 filed Apr. 14, 2017 which is a Continuation in Part application of a U.S. patent application Ser. No. 14/583,792 filed on Dec. 29, 2014, the entire contents of both of which are hereby expressly incorporated by reference.

### TRADEMARKS DISCLAIMER

The product names used in this document are for identification purposes only. All trademarks and registered trademarks are the property of their respective owners.

### FIELD OF THE INVENTION

The present invention disclosed herein relates generally to the field of system having an over-the-counter (OTC) medical devices and an AI/machine learning based computer implemented platform in conjunction with a “smart virtual assistant”, which utilizes the medical device/s to collect this personal heath data via sensors and interrogates the data and with the assistance of their doctors, provides for personalized better patient outcomes. This data in the cloud along with the mobile application provides for an AI powered smart virtual assistant (SVA) which using pre-defined algorithms derived by physician recommendations, triggers the execution of a Q&A session with the patient at his home, and provide this initial answers back to the physician, to provide for just in time care, and early diagnosis for better patient outcomes. Further this invention, in particular, to various embodiments comprising methods, a system, devices and computer media, using portable low level laser technology (LLLT) devices that are pre-programmed with clinical strength doses, and used to provide pain relief caused by the peripheral neuropathy.

### BACKGROUND OF THE INVENTION

Arthritis and osteoporosis are the most common joint pain diseases, and they affect millions of people each year. A patient may suffer from arthritis in a variety of locations on their body. Arthritis may impair a daily routine of the patient. Such impairment can affect the ability of a patient to work or enjoy leisurely activities. Treatments for the pain associated with arthritis comprise medications, injections, and creams. Symptoms of arthritis comprise pain, swelling, inflammation, limited range of motion, and redness. Medications for treating the pain include acetaminophen, (e.g., Tylenol®), NSAIDS—non-steroidal anti-inflammatory drugs (e.g., Advil®, Motrin®, Aleve®), and tramadol.

Use of acetaminophen can cause liver damage, especially if the patient consumes alcohol. Acetaminophen may also affect the efficacy of other medications taken by the patient. Side-effects of non-steroidal anti-inflammatory drugs include gastric ulcers, cardiovascular problems, and gastrointestinal bleeding. Tramadol may cause nausea and constipation. A patient may try creams as an alternative to, or in addition to, medications. Side effects of creams include irritation or redness on the application site of the cream. Both medications and creams have undesirable side-effects.

Further, patients with diabetes sometime bears a pain caused by the peripheral neuropathy, which is a disease caused by lack of blood flow to the extremities such as feet and hands. Peripheral neuropathy is a nerve damage caused by chronically high blood sugar and diabetes which leads to numbness, loss of sensation and sometimes pain in feet, legs or hands.

### An Introduction to LLLT (Low Level Laser Therapy)

Low level laser therapy (LLLT) is a non-invasive treatment that has been used for many years to relieve pain from sports injuries, joint related arthritic illnesses, neck and back pain, shoulder, wrist, knee and ankle related joint pains or many other type of pains caused due to other diseases such as arthritis, peripheral neuropathy etc.

Low levels of laser energy have a non-thermal, bio-stimulative effect on biological tissues. The therapeutic application of low level laser energy, frequently known as low level laser therapy (LLLT) produces beneficial clinical effects in the treatment of musculoskeletal, neurological and soft tissue conditions. LLLT is non-invasive and avoids the potential side effects of drug therapy. More specifically, LLLT delivers photons to targeted tissue, penetrating the layers of skin to reach internal tissues to produce a specific, non-thermal photochemical effect at the cellular level.

Low level laser therapy (LLLT) is the application of light (usually a low power laser or LED/VCSEL in the range of 10 mW-500 mW power) to a pathology (e.g. skin surface of an afflicted joint) to promote pain-relief, reduce inflammation and promote tissue regeneration. The light is typically of a narrow spectral width in the red or near infrared (NIR) spectrum (600 nm-900 nm), with a power density (irradiance) between SmW-500 mW/cm². It is typically applied to the injured anatomical area for a few minutes or so, a few times a week for several weeks. Unlike other medical laser procedures, LLLT is not an ablative or thermal mechanism, but rather a photochemical effect comparable to photosynthesis in plants whereby the light is absorbed and exerts a chemical change.

### LLLT is Therapeutic in Two Ways for Pain Relief

1. Anti-inflammatory. When cells are stressed (e.g. during states of arthritis, osteoporosis, sports injuries, pain caused due to peripheral neuropathy), nitric oxide (NO) inhibits oxygen consumption by mitochondrial cytochrome c oxidase. This reduces production of ATP and causes oxidative stress leading to increased inflammation and reduced production of ATP. LLLT displaces nitric oxide (NO) from cytochrome c oxidase thereby reducing inflammation and restoring ATP production, relieving pain and helping tissues heal more quickly. LLLT reduces oxidative stress by applications to injuries of light of suitable wavelength, sufficient irradiance and time of exposure to cause cytochrome c oxidase to displace mtNO, thereby reducing oxidative stress and increasing ATP production. A cascade of downstream metabolic effects lead to a reduction in inflammatory markers including prostaglandin E2, interleukin 1p and tumor necrosis factor α.

2. Analgesia. LLLT creates a nerve block. Higher irradiance/energy treatments can induce an analgesic effect by disrupting fast axonal transport in small diameter fibers, in particular nociceptors. This temporary (reversible) inhibition of A-delta and C fiber transmission reduces tonic peripheral nociceptive afferent input and facilitates reorganization of the modulation of synaptic connections. Repeated treatments lead to a reduction in central sensitization.

In LLLT, one or more laser beams allow for the use of a carefully selected wavelength, coherently directed to specific tissue/cells, that provides energy to selectively stimulate processes in living cells. This process can help in increasing blood flow, excite cell activity and intensify inter-cell communications. Laser light has been used to effectively treat joint related musculoskeletal pain caused by illnesses including: tendonitis (back pain, knee tendonitis, hand tendonitis, Achilles tendonitis); tennis elbow; carpel-tunnel; arthritis; osteoporosis; plantar fasciitis; and tissue healing from sports injuries and bursitis. Further, the laser light or an LLLT can be proved effective over the pain caused by the peripheral neuropathy due to the high blood sugar and diabetes.

LLLT for ‘peripheral Neuropathy’ can also comprise a specific type called “non-invasive laser acupuncture” comprising the application of low intensity laser radiation (i.e., non-thermal intensities) to classical meridian points or trigger points. Recent scientific studies have demonstrated the therapeutic efficacy of treating the following disorders with non-invasive laser acupuncture: myofascial pain (power is greater than 10 mW, and a dosage of 0.5 Joules per point); post-operative nausea and vomiting (power is greater than 10 mW and a dosage of 0.3 Joules per point); and chronic tension and headaches (see Baxter, G D et al. Clinical Effectiveness of Laser Acupuncture: A Systematic Review. J Acupunct Meridian Stud 2008; 1(2): 65-82).

Until now, the industry has been using either invasive drugs or large expensive laser machines where patients need to be treated clinically with nurse supervision, causing inconvenience and repetitive expenses. More recent inventions comprise hand-held LLLT devices that require the user to treat themselves. These devices for use at home do not deliver clinical strength accuracy or therapeutic efficacy; and they are highly inconvenient because of the lack of device ergonomics and the nature of treatment makes it difficult for the user to apply the device to the user's body in hard to reach areas. Consequently, current solutions available today are comparatively less effective than the present invention of LLLT wraps that are able to encircle and stay in position on a user's body without the user having to hold the device in position during the treatment.

And these prior art hand held LLLT devices do not accurately provide the required treatment power density in joules/cm², due to the following factors. 1. The prior art devices do not treat below the skin surface, whereas the present invention provides both surface and deep tissue treatment options and laser light doses. 2. The prior art devices do not provide a targeted dose at a magnitude equivalent to a LLLT device in a clinical facility, and neither are they pre-calibrated for a specific disorder—e.g. arthritis versus acute injury or pain caused due to peripheral neuropathy. 3. Due to the inherent nature of handhelds, the user must hold the device over the target area with a constant pressure for up to 10 minutes duration, which is physically difficult to do; but the present invention's wraps are hands free devices. 4. Current solutions are complex and most handhelds are inconvenient and uncomfortable to use due to the hard edges on the skin surface contact areas, thus making them less efficient because they do not conform to the body contour, thus easily missing the target areas, especially when self-treated by patient at home. 5. As evidenced by laser acupuncture, the laser dosage needs to be applied at an exact location, and for the doctor recommended duration, for the patient to realize the therapeutic benefits: and the present invention delivers this with precision due to the wearable, hands free, wrap with specifically positioned laser diodes that are preprogrammed to emit a clinical strength dosage.

### Other Portable Phototherapy Devices

The prior art discloses a few portable, phototherapy devices, such as United States Published Patent Application No. 2008/0255640, filed L by Kipp et al., which discloses a portable phototherapy device for treating skin conditions that is packable and can treat various parts of the body. However, Kipp et al. discloses a device that is rigid, uses an ultraviolet blub, and has only one setting. Kipp et al. does not disclose a flexible and pre-programmable phototherapy device.

Also, US Published Patent Application No. 20110144727, filed by Mellen-Thomas Benedict, claims a device for all solutions to use on all body parts, but it does not solve or alleviate the symptoms of any particular malaise, nor does it show sufficient therapeutic efficacy as compared to the LLLT device of the present invention

Similarly, also in the field of portable phototherapy devices, U.S. Pat. No. 6,312,451 B1, filed by Jackson Streeter, discloses a low-level laser therapy apparatus that treats many conditions, but it does not provide the modality for home self-care for patients to use at their convenience in their home, and is it complicated to use and requires a mandatory clinician to treat the patient.

Typically, persons wishing to benefit from phototherapy must go to a spa or a professional health provider/clinic. This is because the phototherapy devices currently available are complicated-to-use and bulky and large. Additionally, until recently, a professional was needed to program and monitor a phototherapy session because optimal parameters, such as wavelength range, relative distribution of the wavelengths within the range (spectrum), time interval for continuous exposure, time interval between two continuous exposures, time rate of energy delivered, accumulated energy density for exposures, and body component(s) irradiated, were not yet available. Now, many of the optimal parameters are better understood. For example, U.S. Pat. No. 6,524,329, issued to Mellen-Thomas Benedict discloses a method of illuminating body components that provides some beneficial treatment.

Still, the complexity of the current laser devices that are used for self-treatment, the inherent in-efficacy of the devices, the dependence on another individual for treatments, and the huge costs to purchase, has made it prohibitive to enable over-the-counter home self-care for patients suffering from joint pain and inflammation, especially due to peripheral neuropathy.

Further, there is not any prior art that may disclose a complete and well defined/maintained system with a compact LLLT wrap devices to monitor and treat joints or other part of body bearing pain due to injuries or any other diseases such as arthritis, osteoporosis or a pain caused due to peripheral neuropathy by use of low level laser emission of desired fixed frequency for a fixed amount of pre-defined time over the area of pain. Real time monitoring and collection of data from the LLLT wrap devices with an integrated mobile “smart virtual assistant” (SVA), over the cloud server accessible by a computer implemented platform to allow doctors, caregivers or loved ones to track and monitor the real time pain condition of the patient which is nowhere being taught by any of the prior art.

Thus, there is a need to provide a system for monitoring and treating a pain caused by peripheral neuropathy, arthritis, osteoporosis or any other muscles pain using the low level laser therapy, where the system having a LLLT device with pre-programmed clinical strength doses, completely portable with a battery powered, wirelessly enabled, electrical circuit embedded within an orthopedic wrap-brace to treat pain in a human while providing joint support. It should be pre-calibrated to treat a specific joint for optimal pain relief and/or inflammation reduction, and able to be operated as the user engages in their normal routine. The present invention provides a LLLT wrap-brace device with these features due to the specific ergonomic fit of the wrap-brace, as well as the accuracy of the LLLT treatment delivery method to the injured area, thus bringing the clinic to the home and making it incredibly simple to use and treat the hard to reach joint pains. Further, there is a need of a system where the real time body data with pain condition of the patient is monitored by the LLLT device is stored over the cloud by the system which is accessible using the computer implemented AI/machine learning, and ‘smart virtual assistant’ powered platform that may allow doctors of other family members of the patient to monitor the health condition of the patient. Moreover, there is a need of a system, where the AI/machine learning powered platform may work as a smart assistant for the patient when he/she feels to have a care administered by the personal physician, at home, at the point of care, and at patient's convenience.

### SUMMARY OF THE INVENTION

The present invention comprises various types of low level laser therapy (LLLT) wrap devices, each designed to fit around a specific anatomical area of a human body (e.g. neck and shoulders, knee, foot and ankle, back, wrist, and elbow), and their method of use, for the treatment of pain and inflammation and to promote tissue regeneration in a human. Each LLLT wrap device is lightweight, hands-free (once in position on the user's body and activated), and completely portable so that the user may wear the wrap while continuing their normal routine. And each LLLT wrap device is pre-calibrated to deliver a clinical strength dosage of infrared and/or near infrared (e.g. red) light in the wavelength from 630 nm to 904 nm that is specific to the medical disorder and/or the user anatomical body part. In an embodiment, all of the laser diodes are of the same type and the user merely activates the device. In another embodiment, the user selects a type of treatment (deep pain versus surface pain) and then activates the power to the laser diodes.

LLLT Wrap Shape and Components: The various types of LLLT wrap device of the present invention comprise a LLLT wrap shaped for encircling a particular area of a user's body, such as: arm: leg; hand: low back; knee; ankle-foot; hand-wrist; neck-upper back-shoulders; and elbow-forearm. But, it is noted that the present disclosure covers a LLLT wrap of any shape for use in relieving pain and/or inflammation associated with a musculoskeletal injury and/or medical condition in a human.

And the wrap device may comprise a variety of types of materials (e.g. rubber/neoprene/cloth/resin) to make it flexible to wrap tightly round a user's body, while providing enough stiffness to provide structural support so the wrap can also function as an orthopedic support brace. When the wrap device is in place on the user, the laser diodes are automatically positioned over the target areas to be treated, and thus resulting in a significant therapeutic treatment modality. Thus, in another embodiment, the present invention comprises a LLLT wrap device for delivering non-invasive laser acupuncture by delivering targeted laser beams to acupuncture meridian points and/or trigger points in a user's body and at a sufficient power and dose to be therapeutically effective against the disorder being treated. For example, in the knee wrap, acupuncture laser treatment is delivered to various points surrounding the patella (medial, lateral, proximal, distal, posterior, and anterior).

Each device is also a unisex wrap that comes with different sizes (S/M/L/XL) to fit all user-patients; and a fixation means for attaching the wrap securely to the user's body (e.g. Velcro-like straps, hooks, snaps, etc.).

Portable: The various embodiments of the LLLT wrap of the present invention further comprise a lightweight (e.g. 150-200 g) and completely portable device due to an embedded electrical circuit comprising: a rechargeable and replaceable battery; a central processing unit; a wireless transceiver; a display for inputting commands; a power switch for automatic shutoff; a plurality of sensors including laser safety sensors, and/or patient monitoring sensors to monitor temperature, blood pressure, EKG, blood glucose level, body dietary/nutrition markers (Keton levels, Insulin levels, etc.) & related data and also a motion sensor to test flexibility and pain; and a plurality of laser diodes. Further, the system and LLLT wrap devices of present invention communicates via Bluetooth and WiFI, with the mobile/cloud application platform for the various data centered innovations.

Laser Diodes: The present invention comprises two primary embodiments of laser diodes with each type of LLLT wrap device: 1) all of the laser diodes are of the same type and/or emit the same dose: and 2) two different types of laser diodes are within the wrap to emit either skin surface treatment, or deep surface treatment.

In the first embodiment, each type of LLLT wrap device comprises a plurality of the same type of laser diode, evenly spaced over the treated area (in front and back, or completely encircling, or only covering the top of), wherein each laser diode emits electromagnetic energy (pulsed or continuously) in wavelengths ranging from 630 nm to 904 nm wavelength, with a mean power output during the total treatment of laser energy dosage from about 1 joule/treatment point to about 20 joules/treatment point. The actual power emittance of the laser diodes is pre-programmed to deliver a dose prescribed by clinicians for a specific body part and/or medical condition, and then to automatically shut-off after the prescribed dose is emitted.

In the second embodiment, the user selects from the mobile app on their smartphone or on the LLLT device display whether to receive treatment for surface pain or deep tissue pain. Hence, the LLLT wrap device comprises a plurality of two different types of laser diodes, one type for treating surface pain (i.e. by emitting electromagnetic energy in the 630 nm to 670 nm wavelength with red light visible lasers), and one type for treating deep tissue pain (i.e. by emitting in the 780 nm to 904 nm which are known as infrared—invisible wavelength emitters). The two types of laser diodes are co-located, or are located on the LLLT wrap at separate locations specific to the treatment protocol (e.g. per FIG. 10), such as in line with acupuncture meridian points or trigger points.

Medical Disorder: The LLLT wrap devices are pre-programmed to treat a specific user anatomical body part (e.g. knee, low or upper back, neck and shoulders, wrist, elbow, foot-ankle, etc.) and/or to provide pain relief, inflammation reduction, and/or tissue regeneration for a specific medical condition, such as by way of non-limiting examples: tendonitis (back pain, knee tendonitis, hand tendonitis, Achilles tendonitis); tennis elbow: carpel-tunnel; arthritis; osteoporosis; plantar fasciitis, peripheral neuropathy, and tissue healing from sports injuries and bursitis. The pre-programmed dose comprises a set amount of energy density, duration, and intensity to be delivered to specific points on the user's body, and then the device automatically shuts-off. The pre-programmed dose is computed from medical research shown to provide the most beneficial therapeutic outcome for using LLLT and/or non-invasive laser acupuncture therapy.

LLLT Wrap System: Each type of LLLT wrap device may further comprise a computer program product (e.g. mobile application) or an AI and/or Machine Learning powered computer implemented platform accessible via a user electronic computing device (e.g. smartphone, laptop, tablet, etc.) for transmitting and receiving patient data, treatment protocols and history of treatments, etc. to produce a LLLT wrap system. In an embodiment, the mobile application automatically syncs (pairs) with the LLLT wrap via a Bluetooth chip in both the user's device and the wrap's transceiver unit, and is thus able to transmit treatment and sensor data from the wrap device to the user's electronic computing device, and/or user operating commands from the user device to the wrap. The mobile application also enables the transmission from the LLLT device to the user's electronic computing device of one or more of the following: user self-reported pain data that they input into the LLLT device display; patient monitoring and sensor data (e.g. blood pressure, body temperature, blood glucose level, body movement data, body dietary/nutrition markers (Keton levels, Insulin levels, etc.) & related data etc.); history of LLLT device usage; etc. This data can also be wirelessly transmitted from the computer implemented platform of present invention to a cloud storage, and vice versa.

According to one embodiment, the present LLLT wrap system includes a AI/machine learning ‘smart virtual assistant’ powered computer implemented platform capable of being accessed by the user electronic computing device that accesses real time data from the cloud storage where it is being stored by the LLLT wrap devices, and using the machine teaming smart algorithm, notifies the family and personal physician of the patient in case of any extremities detected by the LLLT wrap device. According to one embodiment, the AI/machine learning PSA algorithm of present LLLT wrap system analyzes the real time collected data and pain condition of the user by the LLLT wrap devices and accordingly changes the intensity of the laser light being emitted by the LLLT wrap device.

According to one embodiment, the AI/machine learning PSA powered computer implemented platform of present invention behaves as a smart assistant for the patient, where the patient may access the platform using any user computing device. When the user feels the need to have care administered by the personal physician, the platform, based on pre-determined set conditions, launches a screening interactive questionnaire where the patient is asked to answer specific questions that the personal physician would ask when interviewing the patient for the first time.

According to one embodiment, the computer implemented system of present LLLT wrap system activates the smart virtual assistance based on the triggers specific to patient set by the personal physician which takes the patient through the Question & Answer session over the platform via voice on the phone or via text, which further records and stores it over the cloud. According to one embodiment, the computer implemented platform of present system is further embedded with and/or compatible with other healthcare and/or interactive platform such as Amazon Alexa, Apple Siri, Google Assistant etc. that records the answers of the patient and directly stores it over the cloud for the personal physician or a family members, via the integrated AI and/or machine learning powered computer implemented laser wrap platform.

Therapeutic Effect: The LLLT wrap devices of the present invention produce beneficial clinical effects in the treatment of pain and/or inflammation and/or to promote tissue regeneration for musculoskeletal, neurological and soft tissue conditions, while being non-invasive and avoiding the potential side effects of drug therapy. More specifically, each type of LLLT wrap device of the present invention delivers photons to targeted tissue, penetrating the layers of the user's skin to reach internal tissues to produce a specific, non-thermal photochemical effect at the cellular level. Pain associated with medical disorders treatable using the LLLT wrap device comprises, by way of non-limiting examples, pain associated with: tendonitis of the back, knee, hand, and Achilles tendon; tennis elbow; carpel-tunnel; arthritis (rheumatoid and osteoarthritis); osteoporosis; plantar fasciitis; bursitis; muscle and/or tissue inflammation and damage from acute and chronic injuries, and a pain caused due to peripheral neuropathy caused due to high blood sugar level or diabetes.

In particular, the analgesic effects from each treatment with the LLLT wrap device lasts for about 48 hours. There is also significant reduction of inflammation equal to or better than non-steroidal anti-inflammatory drugs (NSAIDs) within 2-12 hours of treatment. And the healing time of chronic tendinopathies is reduced by about 70%. And there are no adverse side effects from the treatments. In one embodiment, optimal pain relief is achieved with a plurality of treatments, such as the recommended treatments of 5-10 times a week for a period of 5-12 minutes per session is applied, depending on the joint being treated.

### Method of Use

Usage: Each type of LLLT wrap device is pre-calibrated for the amount of dosage it emits (e.g. duration, power, etc.) using specifically required laser wavelengths (nM) to achieve the desired tissue penetration depth for optimal treatment as well as further controlled by the synced and/or coupled AI/machine learning powered computer implemented platform with a smart virtual assistant that monitors real time user data and pain level or other body vital data and determines using the machine learning algorithm, the best suited and/or required frequency, duration and intensity of the laser for the best treatment of the user, while facilitating the physicians recommendations and patient point of care. Due to the “pre-calibration”, the patient merely places the specific type of LLLT wrap device over or around their afflicted anatomical area, turns on the power button, and the device treats the area by delivering the optimum number of photons required for the type of joint or anatomical area and/or the type of medical disorder. The same power button acts as the “Pause-Restart” button if required, such as if the user needs to take a break. The device shuts-off automatically after the programmed pre-determined and calibrated treatment time is achieved. In one embodiment, depending on the joint being treated, the self-care treatment is most accurate for optimal pain relief when recommended treatments of 5-10 times a week for a period of 5-12 minutes per session is applied. According to one embodiment, the computer implemented platform further allows user to switch On/Off the LLLT wrap device using the user computing device or the platform may itself switch On/Off the device by analyzing the real time body vital data of the user.

These pre-programmed treatment protocols provide for proven optimal healing and pain-relief at home or work, at the point of care, for timely intervention. The LLLT laser wrap device allows patients to treat the hard-to-reach target areas that cause joint pains. Furthermore, each type of pre-programmed, calibrated LLLT wrap device ensures optimal accuracy of the treatments to the afflicted areas without a clinician's assistance, thus making it a simple to use outside of a clinical facility.

During and/or after each treatment session, the LLLT wrap device wirelessly transmits the user's history of treatment sessions, self-reported pain measurement levels, pain medication intake, device's patient monitoring sensor data (e.g. vital signs data, such as by way of non-limiting examples: blood pressure, body temperature, blood glucose level, body movement data, body dietary/nutrition markers (Keton levels, Insulin levels, etc.) & related data etc.) to the cloud server from where it can be accessed by the computer implanted AI/machine learning powered platform with a smart virtual assistant and thus by any user of interest over the user computing device.

In one embodiment, the method of use of the LLLT wrap device comprises the following steps: the user cleans the anatomical area so that their skin is conducive to treatment; the user inputs their selection of the type of treatment (i.e. treating skin surface pain, or deep pain requiring penetration of the laser light into the tissue) on their mobile device display, or on the LLLT device display; the user adjusts the laser diodes power output to their comfort level (e.g. one-third, one-half, or one hundred percent of the maximum power output) and pushes the power button; the LLLT wrap device emits a pre-calibrated does of irradiation based on the type of wrap (knee, neck, etc.) and/or the medical condition, after which it automatically shuts off. The user can pause the LLLT wrap device at any time to attend to other things and resume until completion of treatment. Before, during, or after treatment, the user can input into the mobile application on their electronic computing device, or the LLLT wrap device display, their pain level and/or their consumed pain medication.

In summary, the various type of LLLT wrap devices disclosed herein enable patients to have an affordable, hands-free, easy-to-use, clinical strength, pre-calibrated, worry-free, pain-relief medical device for use at home, with the added ability to track the patient's vital signs data through monitoring sensors embedded in the devices. Thus, the LLLT wrap devices of the present invention enable self-care, allow loved ones to monitor the patients on their mobile phones, and empower the patient to improve their lives tremendously. This invention is also a portable, hands-free and wearable LLLT wrap, as well as an orthopedic support brace, that provides a pre-calibrated laser energy power dosage (e.g. within the range of 5 mW-500 mW) that is administered automatically, for specific durations and treatment depth, to the specific desired area. This targeted treatment ensures maximum therapeutic efficacy through ergonomic fit so as to effectively treat the specific joints and thus provide optimal joint-pain relief. It also saves the user time and money by not having to commute to a clinician's office for treatment, and pay for the clinician's services.

According to one embodiment, the present invention provides a system having a various types of LLLT wrap devices having plurality of various sensors to monitor and send the vital data to the cloud server in real time. The various LLLT wrap devices are the wraps for different body parts such as hand, arm, foot, ankle or any other body part to treat the pain using the laser therapy. The LLLT wrap devices includes a plurality of laser diodes of different frequencies and intensities to emit the laser light of a wavelength suitable for the treatment of specific pain. The system further includes a computer implemented platform with a ‘smart virtual assistant (SVA)’ coupled with the cloud server which is powered by the Artificial Intelligence or a Machine learning Algorithm that monitors the vital body data of the user in real time and automatically interrogates the patient as a first line of physician derived questions and answers, accordingly notifies the responsible healthcare personnel and a family members of the user about the real time pain condition. Further, the computer implemented platform of present invention behaves as a smart assistant to the user which analyzes present and past medical history of the user/patient and based on the triggers specific to the patient set by the personal doctor, the platform prepares a questions for the patient that generally the physician asks when consulted for the first time. The user may answer that question directly using voice/text in the phone or by using any other smart device embedded within the present system and platform such as the Amazon Alexa, Apple Siri, Google Assistant etc.

### BRIEF DESCRIPTION OF THE DRAWINGS

The above and other features, aspects, and advantages of the present invention will become better understood with regard to the following description, appended claims, and accompanying drawings where:

FIG. 1A is a sectional view of an open wide belt (1) for treating a variety of body areas (e.g. neck, bicep, calf, quadricep, etc.) containing multiple lighted lasers (2) with a Velcro-like system (3) to hold the belt in place.

FIG. 1B is a sectional view of a closed belt (1) containing multiple lighted lasers (2) with a Velcro-like system (3) holding the belt in place.

FIG. 2 is a sectional view of an open wide belt (1) containing multiple lighted lasers (2) with a Velcro-like system (3) where subject (4) is about to wear the belt for treating low back pain.

FIG. 3 is a sectional view of a closed belt (1) containing multiple lighted lasers (2) with a Velcro-like system (3) where subject (4) has strapped the belt in place around his waist (5)

FIG. 4A is a sectional view of an open wide Knee Wrap (6) containing multiple lighted lasers (7) with a Velcro-like system (8).

FIG. 4B is a sectional view of a closed wide Knee Wrap (6) containing multiple lighted lasers (7) with a Velcro-like system (8) holding the closed secure fit.

FIG. 4C is a sectional view of an open wide Knee Wrap (6) containing multiple lighted lasers (7) with a Velcro-like system (8) being applied on the user's Knee (9).

FIG. 4D is a sectional view of a closed wide Knee Wrap (6) containing multiple lighted lasers (7) with a Velcro-like system (8) showing the closed secure fit on user's Knee (9).

FIG. 5A is a sectional view of a closed Ankle Wrap (10) containing multiple lighted lasers (11) with a Velcro-like system (12) showcasing a secure fit.

FIG. 5B is a sectional view of a closed Ankle Wrap (10) containing multiple lighted lasers (11) with a Velcro-like system (12) showcasing a secure fit around the user's ankle (13).

FIG. 6A is a sectional view of an open Hand Wrap (14) containing multiple lighted lasers (15) with a Velcro-like system (16) used to secure the hand wrap (14).

FIG. 6B is a sectional view of a closed Hand Wrap (14) containing multiple lighted lasers (15) with a Velcro-like system (16) showing the securely closed hand wrap (14).

FIG. 6C is a sectional view of an open Hand Wrap (14) containing multiple lighted lasers (15) with a Velcro-like system (16) where the user is about to wrap it around their hand (17).

FIG. 6D is a sectional view of a closed Hand Wrap (14) containing multiple lighted lasers (15) with a Velcro-like system (16) showing the Hand Wrap (14) securely fastened on the user's hand (17).

FIG. 7A is a sectional view of an open Neck & Shoulder Brace Wrap (18) containing multiple lighted lasers (19) with a Velcro-like belt system (20).

FIG. 7B is a sectional back view of a closed Neck & Shoulder Brace Wrap (18) containing multiple lighted lasers (19) with a Velcro-like belt system (20) used to securely wrap the shoulder brace around the user's shoulders (21).

FIG. 8A is a sectional view of an open Elbow Brace Wrap (22) containing multiple lighted lasers (23) with a Velcro-like wrap & belt system (24) showcasing the secure fit.

FIG. 8B is a sectional view of a closed Elbow Brace Wrap (22) of FIG. 8A shown being worn securely on subject's elbow (25).

FIG. 9 a schematic diagram of an exemplary electrical circuit embedded within each LLLT wrap device that is used to power the device and wirelessly transmit user data to a mobile application installed on a user electronic computing device.

FIG. 10 is top perspective view of a knee LLLT wrap device unfolded, and comprising two sets of dual lasers: surface diodes emitting red light between 630-670 nm, and deep penetration laser diodes emitting infrared light between 780-904 nm.

FIG. 11 is a flowchart of the method of use of the LLLT wrap device and the mobile application synced with the LLLT wrap device.

FIG. 12 is a schematic block diagram of a user electronic computing device for use with the mobile application of FIG. 11 and having the mobile application of the present invention installed thereon.

FIG. 13A shows one another embodiment of a flexible wrap of present invention capable of being wrapped around any part of the body.

FIG. 13B shows turned view of the same embodiment of the flexible wrap disclosed in FIG. 13A.

FIG. 13C, FIG. 13D and FIG. 13E shows flexible wrap disclosed in FIG. 13B wrapped over the body of the user.

FIG. 14 shows a data flow diagram of present LLLT wrap system having a different wrap devices and the computer implemented AI/ML powered platform with smart virtual assistant (SVA).

FIG. 15 shows a block diagram of present system with an integrated virtual assistant within the platform.

### DETAILED DESCRIPTION OF EXEMPLARY EMBODIMENTS

### Types of LLLT Wrap Devices

The present invention includes the following exemplary types of LLLT wrap devices listed below: low back; knee; ankle-foot; hand-wrist; neck-shoulder; and elbow.

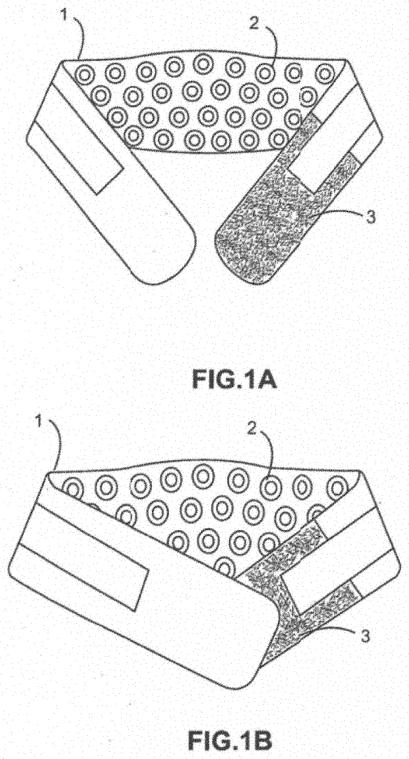

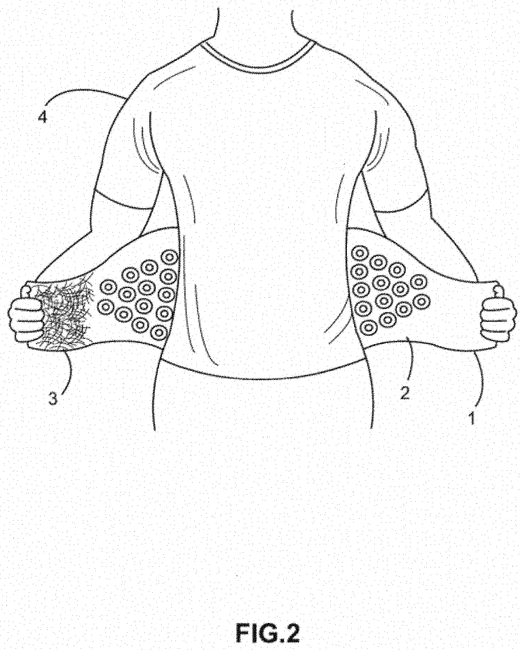

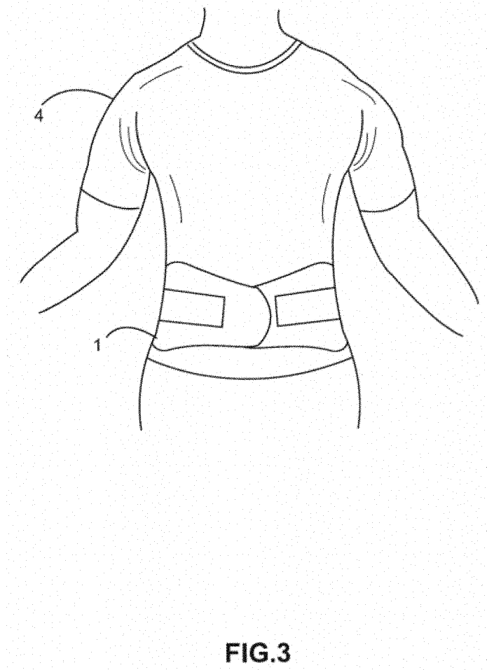

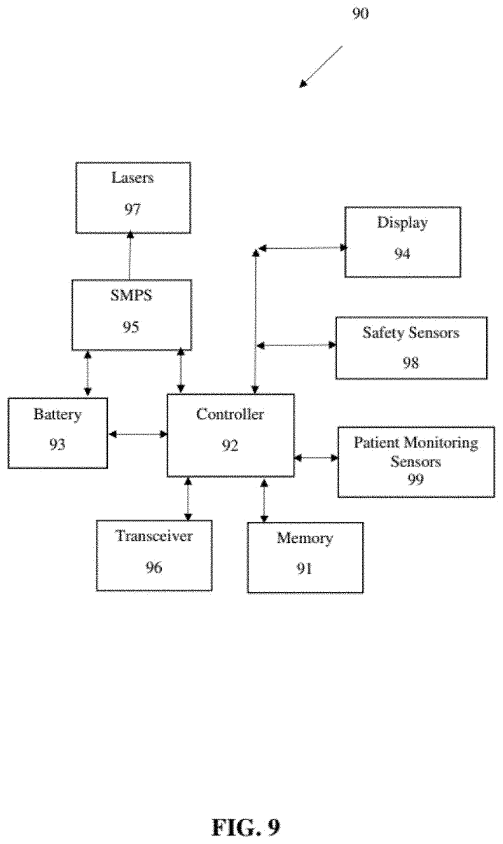

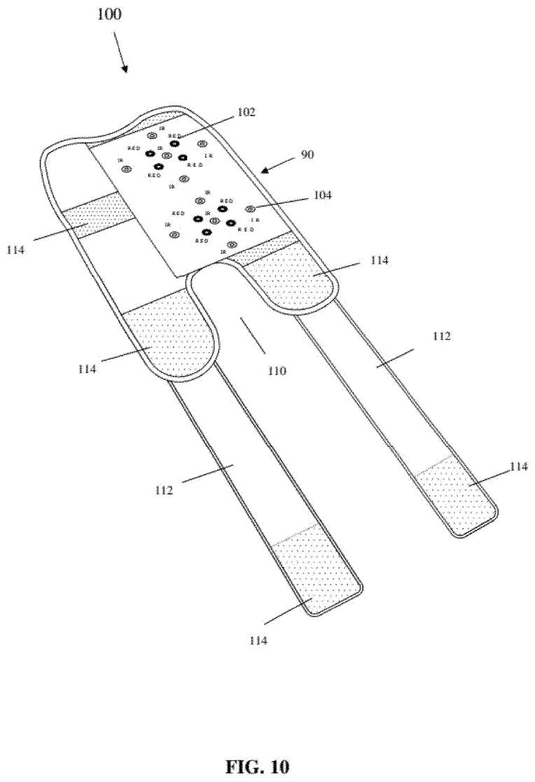

Low Back LLLT Wrap: LW-Back-1000-Laser-wrap device (FIG. 1A, 1B, 2, 3; item 1) placed on the human upper or lower back (FIG. 2, 3; item 4)—for treating musculoskeletal back pain associated with the peripheral neuropathy, arthritis, osteoporosis, muscle strain-sprain, etc. In the embodiments exemplified in FIGS. 1A, 1B, 2, 3, each hand-wrist LLLT wrap device 1 further comprises: a plurality of evenly spaced laser diodes 2; a Velcro-like member or tab 3 on both opposing ends of the device to affix the device to the user's low back; and an embedded portable electrical circuit not shown (e.g. FIG. 9, 90).

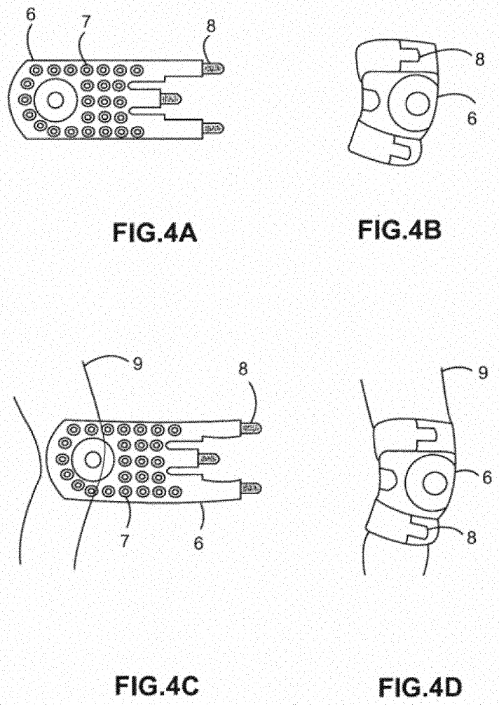

Knee LLLT Wrap: LW-Knee-1000-Laser-wrap (FIGS. 4A-4D; item 6) for the human knee (FIGS. 4C, 4D; item 9)—for treating musculoskeletal knee pain associated with arthritis (rheumatoid and/or osteoarthritis), osteoporosis, muscle strain-sprain, and acute and chronic injuries to the various tendons and ligaments, bone, and cartilage of the knee. In the embodiments exemplified in FIGS. 4A-4D, each knee LLLT wrap device 6 further comprises: a plurality of evenly spaced laser diodes 7 that cover the right and left and back side of the knee, and a single row around a circular opening for the patella-kneecap: a plurality (e.g. three) Velcro-like tabs 8 on parallel extensions of the device to affix the device to the user's knee above, below and behind the knee joint; and an embedded portable electrical circuit not shown (e.g. FIG. 9, 90).

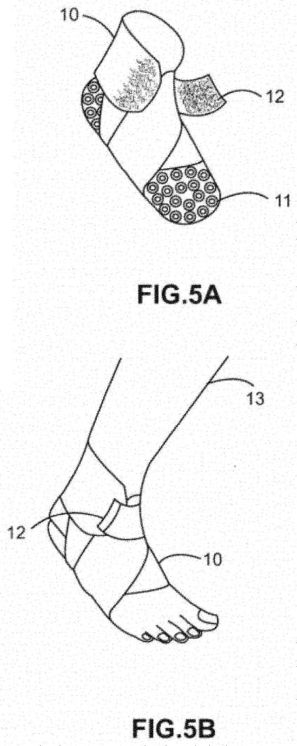

Ankle-Foot LLLT Wrap: LW-Ankle & Foot-1000 Laser-wrap (FIGS. 5A, 5B; item 10) for the human foot/ankle (FIG. 5B, item 13)—for treating pain associated with ankle and/or foot pain, such as peripheral neuropathy caused due to high blood sugar or diabetes, plantar fasciitis, neuropathology (e.g. associated with diabetes), ankle or foot muscle sprains-strains, etc. In the embodiments exemplified in FIGS. 5A and 5B, each LLLT wrap device 10 further comprises: a plurality of evenly spaced laser diodes 11 covering the foot (FIG. 5A) and covering the ankle (not shown FIG. 5B); rectangular-shaped strips encircling the foot arch (which may further comprise laser diodes 11); a plurality (e.g. at least two) Velcro-like tabs 12 on the ends of the strips that wrap around the ankle; and an embedded portable electrical circuit not shown (e.g. FIG. 9, 90) having plurality of sensors including, but not limited to, continuous glucose monitoring (CGM) sensor, motion sensors to continuously monitor flexibility, body dietary/nutrition markers (Keton levels, Insulin levels, etc.) & related data and other vital sensors such as temperature, blood pressure, EKG, etc.

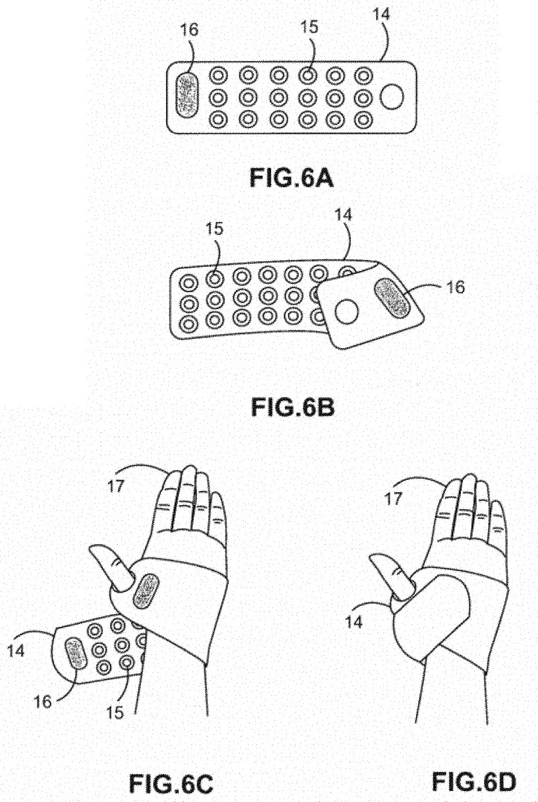

Hand-Wrist LLLT Wrap: LW-Wrist-1000-Laser-wrap device (FIGS. 6A-6D; item 14) for the human hand-wrist (FIGS. 6C, 6D; item 17)—for treating joint pain associated with carpel tunnel, hand and/or wrist sprains, peripheral neuropathy, etc. In one embodiment exemplified in FIGS. 6A-6D, each hand-wrist LLLT wrap device further comprises: a plurality of evenly spaced laser diodes 15; a Velcro-like tab 16 on both opposing ends of the device to affix the device to the hand; a thumb hole to slip the device over; and an embedded portable electrical circuit (not shown) (e.g. FIG. 9, 90).

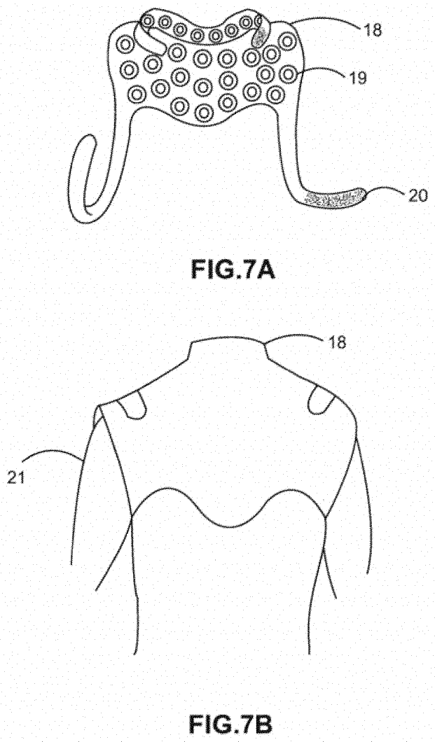

Neck-Shoulder LLLT Wrap: LW-Neck & Shoulder-1000 Laser-wrap (FIGS. 7A, 7B; item 18) for the human neck/shoulder (FIG. 7B; item 21) for treating pain in the user's neck and/or shoulders. In one embodiment exemplified in FIGS. 7A and 7B, each neck-shoulder LLLT wrap device 18 further comprises: a plurality of evenly spaced laser diodes 19 on the inner (i.e. skin side) collar and the inner upper back and shoulder area; a Velcro-like tab 20 on both opposing ends of the device straps that wrap from the bottom of the back side of the wrap under the armpits and affixing to the front outer surface of the LLLT wrap device; and an embedded portable electrical circuit not shown (FIG. 9, 90).

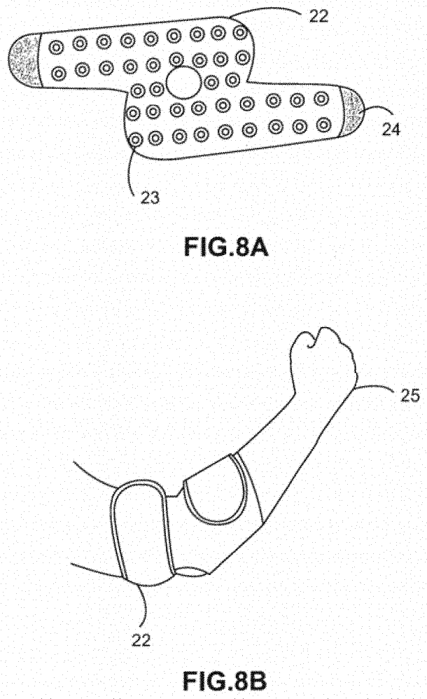

Elbow LLLT Wrap: LW-Elbow-1000 Laser-wrap (FIGS. 8A, 8B; item 22) for the human elbow of arm 25 for treating elbow joint pain, such as associated with arthritis, bursitis, tennis elbow, etc. In one embodiment exemplified in FIGS. 8A and 8B, each elbow LLLT wrap device further comprises: a plurality of evenly spaced laser diodes 23 on the inner (i.e. skin side); a Velcro-like tab 24 on both opposing ends of the device straps for securing each end of the wrap around the user's arm—one upper arm strap and one forearm strap; a center hole for the encircling the elbow; and an embedded portable electrical circuit (not shown)(FIG. 9, 90).

It is further appreciated that the laser diodes shown in FIGS. 1A-8B could readily be replaced with various configurations of surface and deep tissue laser diodes by one of skill in the art, and as exemplified in the knee wrap of FIG. 10.

It is further appreciated that all the above laser wrap devices are fully integrated into the mobile/cloud based AI-machine learning comprehensive data platform with ‘smart virtual assistant’.

Electrical Circuit

FIG. 9 is a block diagram of an exemplary electrical circuit 90 embedded in the wrap-brace of the present invention. Each type of wrap device is embedded with an electrical circuit that is pre-programmed to treatment disorders associated with the particular joint or body part that the type of wrap device covers. Therefore, each type of wrap is pre-programmed to emit irradiation at a set dose (intensity and duration) and to automatically turn off when the dose is completely administered.

The present invention is a battery powered and the battery can be recharged using a 230/110 V charger that will be provided in the LLLT wrap system. The battery may also be replaceable by the user or manufacturer. All LLLT wrap device components, including the printed circuit board (PCB), the battery, the display unit, and controller, are positioned ergonomically and fixed (e.g. sewn, glued, etc.) into the orthopedic wraps to make a sleek form-fitting design that is comfortable to wear by the patient, easily hidden beneath their clothing, while maintaining the treatment efficacy of using the device.

As illustrated, the printed circuit board (PCB) comprises electrical circuit 90 with the following components: a computer memory module/chip 91; a microprocessor or controller 92; a portable power source (battery) 93; a display or graphical user interface (GUI) 94: a power switch 95; a wireless data transceiver, or transmitter, or receiver unit 96; and a plurality of laser diodes 97.

Sensors: The wraps may further comprise various types of sensors 98, 99 embedded within the LLLT wrap device that are connected to the electrical circuit 90, thus powered by the power source 93 and transmitting data wirelessly via unit 96. And/or, at least one type of sensor embedded within the LLLT wrap device is powered by and/or wirelessly transmitting-receiving data independently from the electrical circuit 90 via circuitry connected to and/or within the sensor. By way of non-limiting example, various types of sensors within the LLLT wrap comprise: at least one laser safety-irradiation sensor 98; and/or at least one patient monitoring sensor 99 (see infra).

The computer memory 91 stores pre-calibrated, pre-programmed treatment protocols comprising doses of irradiation (duration and intensity) for at least one protocol, such as surface and/or deep penetration session suitable for the type of wrap, body part, and disorder being treated. It may further record the number of times each type of treatment has been completed by the user. The record may further comprise a time stamp of the date and time of day of the treatment completion. This data may be wirelessly transmitted to the user's electronic computing device, cloud storage, clinician's computer storage, etc.

The microcontroller or processor 92 executes one of the stored programs at a time.

In the exemplified embodiment, the portable power source 93 is a re-chargeable battery (e.g. nickel cadmium battery), that supplies power to the entire electrical circuit 90 and thus enables the wrap to be completely portable. The user is thus able to continue their normal routine while undergoing treatment.

The display or graphical user interface (GUI) 94 displays the selection of treatment protocols and user instructions, and receives user input for selecting the desired protocol (e.g. surface treatment versus deep penetration treatment). Display 94 may also display a clock-timer that counts up or down the pre-programmed treatment duration.

The power switch 95, or switched mode power supply (SMPS), comprises an automatic shut-off mode of power to the lasers when the treatment session is complete, and/or when the sensors indicate a safety alert. The power switch 95 may further emit a sound (e.g. beep) to indicate a power state. For example, a single short duration beep indicates that the power has been turned on in the LLLT wrap; two beeps indicate the end of a treatment session; and a single long duration beep is emitted when a treatment session is activated or in pause mode.

The wireless transmitter or a transmitter/receiver unit 96 sends, and/or sends and receives, data from the wrap electrical circuit 90. All modes of data transmission are wireless, thus unit % comprises a WiFi enabled unit for internet transmissions to any location (e.g. a remote doctor's office computer), and/or a paired short range radio frequency transmission to a co-located user's electronic computing device (e.g. smartphone with mobile application of the present invention installed thereon). In one exemplified embodiment, unit 96 comprises a Bluetooth chip paired with a Bluetooth chip in a user's electronic computing device, but other wireless transceiver units are readily apparent to the skilled artisan.

Laser Diodes

The present invention comprises two primary embodiments of laser diodes with each type of LLLT wrap device: 1) all of the laser diodes are of the same type and/or emit the same dose; and 2) two different types of laser diodes are within the wrap to emit either skin surface treatment, or deep surface treatment, depending on the treatment protocol that the user selects.

When the LLLT wrap device is in-place on a user's body, each laser diode 97 should be aligned to emit a beam substantially perpendicular in to a user's skin surface. The depth of penetration of the beam is dependent upon the type of treatment the user selected (surface pain treatment or deep pain treatment). Each type of LLLT wrap device is specifically designed so that a plurality of irradiation beams enter the user's skin at anatomical locations pre-determined by clinicians and medical research to optimally treat a user's condition (i.e. optimal treatment as defined herein refers to the most therapeutic effective outcome for the reduction of a user's pain, inflammation, etc.) and is due to the location of the laser diodes 97, their level of emittance-intensity and duration, and thus the total dose of irradiation delivered at the afflicted anatomical site.

In one embodiment, each type of LLLT wrap device comprises a plurality of the same type of laser diode, evenly spaced over the treated area (in front and back, right and left side, or completely encircling, or only covering the top of), wherein each laser diode emits between 630 nm to 904 nm wavelengths, with a mean power output during the total treatment of laser energy dosage from about 1 joule/treatment per point to about 20 joules/treatment per point. The actual power emittance of the laser diodes is pre-programmed in to deliver a dose prescribed by clinicians for a specific body part and/or medical condition and then to automatically shut-off.

In another embodiment, as illustrated in the knee LLLT wrap device 100 of FIG. 10, the user selects whether to receive treatment for surface pain or deep tissue pain. Hence, the LLLT wrap device comprises a plurality of two different types of laser diodes, one type 102 for treating surface pain (i.e. by emitting in the 630 nm-670 nm for which are known as red light visible lasers diodes 102), and one type 104 for treating deep tissue pain (i.e. by emitting in the 780 nm-900 nm which are known as infrared laser diodes 104, or invisible wavelength emitters). The two types of laser diodes are co-located, or are located on the wrap at separate locations specific to the treatment protocol. For example, the knee LLLT wrap device 100 of FIG. 10 comprises two sets of four red light emitters diodes 102 for treating surface pain that are arranged within two sets of five infrared laser diodes 104. The two sets of surface diodes 102 and deep penetrating diodes 104 can be arranged on the knee wrap 100 to be situated medial-lateral, or anterior-posterior.

The knee wrap 100 further comprises a substantially rectangular shaped member that the diodes 102, 104 are directly attached to. The rectangular member further resides on a substantially larger rectangular member with a semi-circular cutout 110 for the user's patella to not be covered when the wrap 100 in positioned on the user. Furthermore, one strap 112 extends from both opposing sides of the cutout 110 to wrap around the user's knee—one around the femur—lower thigh and one around the tibia—upper calf. To keep the knee wrap device 100 in position on the user's knee, the wrap 100 further comprises a plurality of fixation members, such as Velcro-like material 114 attached to the rectangular members and to straps 112 (e.g. FIG. 10, Velcro is depicted as dotted patterns).

A variety of different types of laser diodes 97 may be used within the LLLT wrap device, e.g. continuous emission or pulsed emission. Table 1, infra, provides a disclosure on the requirements for any type of laser diode used in the present invention, such as: up to 200 mW in micro-pulses or continuous emission, and energy density of 9-20 joules per minute per centimeter **2; a peak energy emittance per minute of 14/4 joules over the entire afflicted skin area being treated; a coherent beam—meaning that the dose emitted is equal over the cross-sectional area of the laser diode

For example, surface pain treatment may comprise, for example, a plurality of Mitsubishi® laser diode type ML101J23, that emit 658 nm of visible light, with a high-power output 30 mW pulsed. And deep pain treatment may comprise, for example, a plurality of QL80R4S-A/B/D/C/D/E-Z5 laser diodes manufactured by Quantum Semiconductor International Co., Ltd®); and each diode emits 808 nm infrared light wavelength, with an optical output power of 200 mW. It is noted that one of skill in the art would readily know of the type of laser diodes to use with the various LLLT wrap devices disclosed herein.

Pre-calibration of dose: In either embodiment, it is not the total number of joules delivered at a certain skin depth that is important. Instead, the important parameter is the energy density; that is, energy per unit area, more commonly called dose with units of J/cm2. Stated otherwise, the “Energy Density” calculation comprises: power density in units of Watts/cm2 multiplied by treatment time in seconds yields dose in units of Joules/cm2. This is the energy deposited per area of irradiated tissue.

From the depth dose profile, a distinct version of which is necessary for each wavelength, frequency, and power setting as well as for every type of material through which the laser beam will penetrate (skin, bone, soft tissue, fat, etc.), the LLLT wrap electric circuit is pre-programmed to output the intensity and the power density across a desired area. From the power density at a given tissue depth, the dose can be computed: e.g., power density in units of Watts/cm2 multiplied by treatment time in seconds yields dose in units of Joules/cm2.

An exemplary computation of the dosage and total energy (Joules) for a surface treatment versus a deep tissue treatment for a LLLT knee wrap device comprises:

TOTAL ENERGY(J)=Average Power(Watts)×Time (sec)

### Surface Treatment

8 diodes at 5 MW=40 mW of total power
Total joules provided to treatment area (Joules=power in MW*Time)
   10 Joules=0.04 W (40 mW)×250 secs (4.1 minutes)

### Deep Tissue Treatment

10 diodes of 30 MW=300 MW of total power
Total joules provided to treatment area (Joules=power in MW*Time)
   10 Joules=0.3 W (300 MW)×30 secs (½ min)

Energy Density(J/cm²)=Total amount of energy(J)/Irradiation area(cm²)

Example energy density=103/80 cm² (treatment area)=0.125 joules/cm²

### Wrap Sensors

Sensors: The wraps may further comprise various types of sensors embedded within the LLLT wrap device, such as: at least one laser safety-irradiation sensor 98; and/or at least one patient monitoring sensor 99.

Safety-Irradiation Sensors: One or more different types of safety-irradiation sensor 98 may be embedded within the LLLT wrap, such as: a sensor to monitor the amount of laser irradiation dosage being emitted; and/or a sensor to prevent the over-heating of the laser diodes such that the diodes are at risk of being damaged and/or burning the user's skin; and/or a proximity sensor to prevent the laser diodes 97 from turning on unless an object (e.g. a user's skin) is within a fixed distance from the diodes 97 (e.g. about 1 inch or less).

If an unsafe situation occurs, sensor 98 may send an electric signal to the power switch 95 to automatically shut-off the laser diodes 97. Alternatively, or additionally, sensors 98 may display a safety alert on the wrap display 94, and/or wirelessly transmit via the electric circuit wireless transmitter unit % an electronic message to be displayed on a user electronic computing device. It is further noted that the safety sensor features may be built into the laser diodes 97, or separate from the laser diodes 97 but powered by the electrical circuit 90, or having their own source of power and/or wireless transceiver while still being embedded within the LLLT wrap.

User-Patient Monitoring Sensors: The wrap may further comprise sensors 99 embedded in the wrap for monitoring a user's vital signs and wirelessly transmitting the data to the user's electronic computing device, cloud storage, doctor's office computer, etc. Sensor 99 is positioned within the wrap to be in contact with the user's skin when the wrap is in position for treatment. The wrap may comprise more than one type of sensor 99, with each measuring one or more different vital signs. The anatomical position of the wrap may determine which user vital functions are monitored (e.g. a neck—back wrap can be used to monitor pulmonary and cardiovascular conditions; and a knee wrap can be used to monitor neurological conditions). And/or the sensor 99 can measure a vital sign no matter the anatomical location of the wrap, such as user body temperature, heart rate, blood pressure, etc.

In one embodiment, sensor 99 may comprise a patch (e.g. Healthpatch Biosensor manufactured by Vital Connect®) embedded in the wrap to monitor the user's biometric data and wirelessly transmit the data via the electrical circuit 90 wireless unit 96. Biometric data comprises, for example, one or more of: pulmonary (respiratory rate), neurologic (gait analysis, fall detection/severity), cardiovascular (heart rate variability, heart rate, single-lead ECG, contextual heart rate), biometric sensors related to nutrition (Ketones, insulin levels, etc.) and other (step count, posture, body temperature, summarized activity, energy expenditure, stress).

And in another or additional embodiment, sensor 99 may comprise a biosensor tattoo (e.g. Laboratory of Nano bioelectronics by Prof. Wang) imprinted into the skin side surface of a wrap that is able to monitor a user's biometric data, e.g. via the user's sweat, pulse, etc., and wirelessly transmit the data via the electrical circuit 90 wireless unit 96. Biometric data comprises, for example, one or more of: metabolite levels, electrolytes, ammonia, sodium, lactate levels and pH, etc. to measure a user's level of physical activity, and/or a medical condition: glucose level for diabetes, body dietary/nutrition markers (Ketone levels, Insulin levels, etc.) & related data, etc.

In another embodiment, sensor 99 may comprise a disc shaped, metallic sensor connected to the electrical circuit 90 and able to detect a user's body temperature (e.g. MySignals™ by eHealth Medical Development); or a user's pulse rate (e.g. Arduino® sensor).

### Mobile Application and Method of Use

The present invention further comprises a computer program product (e.g. a mobile application comprising a non-transitory computer-readable storage medium) installed on a user's electronic computing device (e.g. smartphone, tablet, laptop, etc.) for wirelessly receiving and displaying data from the wrap electrical circuit (e.g. sensor data, self-reported pain level and/or amount pain medication taken data, timer, etc.), and/or wirelessly transmitting commands to the wrap electrical circuit (e.g. selection of treatment protocol), for example—in order to control the power output ⅓^{rd}, ½, or ⅔^{rd} power.

The mobile app also houses the AI and/or Machine Learning (ML) powered ‘smart virtual assistant (SVA)’ that may be used by the patient to facilitate the first line of questions and answers for physicians based on certain physician recommended triggers, for alerting, intervention and better patient outcomes.

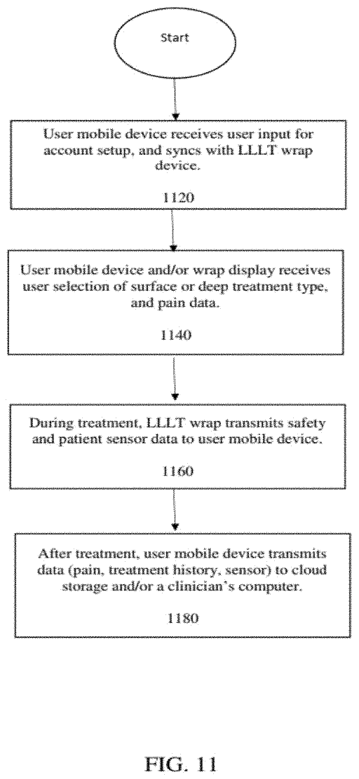

FIG. 11 is a flowchart of steps for using the mobile application 220 of the present invention that is installed on the user electronic computing device 200 to collect user pain data and store it on a cloud account and/or a remote clinicians' computer.

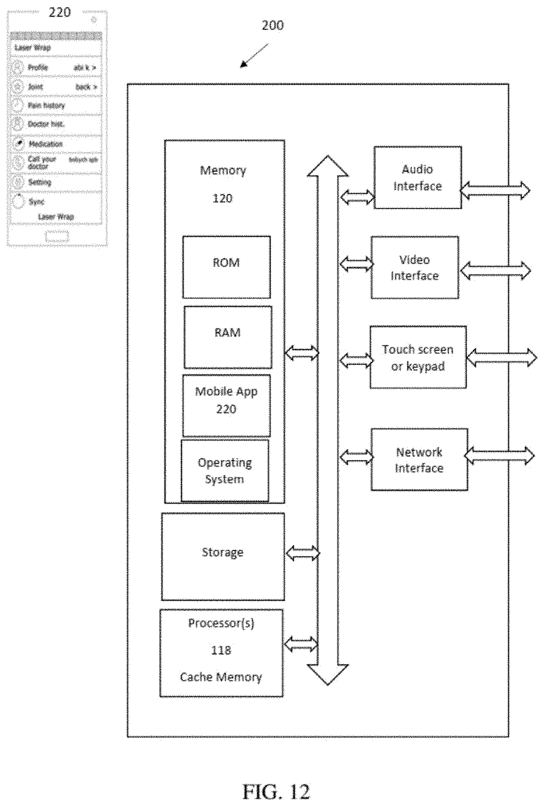

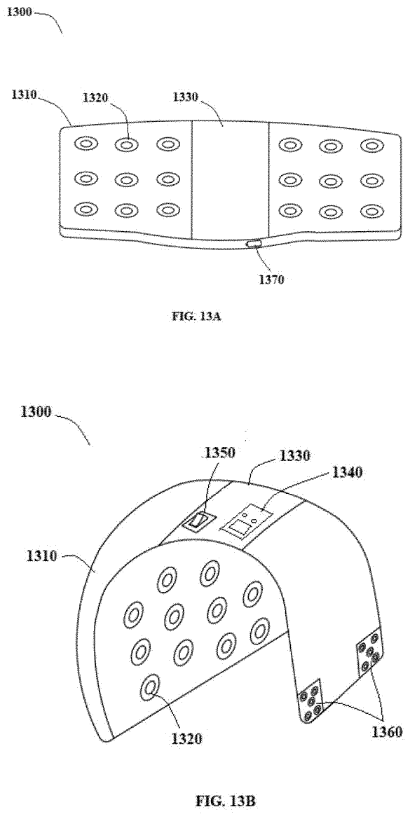

As illustrated in the exemplary embodiment of FIG. 12, a user's electronic computing device comprises the following components: a central processing unit 118, a memory unit 120, that stores machine instructions that when executed by the processor 118, cause the processor 118 to perform one or more of the operations and methods described herein. Processor 118 may optionally contain a cache memory unit for the temporary local storage of instructions, data, or computer addresses. For example, using instructions retrieved from memory 120, the processor 118 may control the reception and manipulation of input and output data between components of the user's electronic computing device. In various embodiments, the processor 118 can be implemented as a single-chip, multiple chips and/or other electrical components including one or more integrated circuits and printed circuit boards.

The processor 118 together with a suitable operating system may operate to execute instructions in the form of computer code and produce and use treatment data. By way of example and not by way of limitation, the operating system may be Windows-based, Mac-based, or Unix or Linux-based; and in particular for smartphones, the operating system comprises one of Android, iOS, and Windows Mobile among other suitable operating systems. Operating systems are generally well known and will not be described in further detail here.

Memory 120 encompasses one or more non-transitory storage mediums and generally provides a place to store computer code (e.g., software and/or firmware) and data that are used by the use electronic computing device. It may comprise, for example, electronic, optical, magnetic, or any other storage or transmission device capable of providing the processor 118 with program instructions. Memory 120 may further include a floppy disk, CD-ROM, DVD, magnetic disk, memory chip, ASIC, FPGA, EEPROM, EPROM, flash memory, optical media, or any other suitable memory from which processor 118 can read instruction, in computer programming languages.

Memory 120 may include various other tangible, non-transitory computer-readable media including Read-Only Memory (ROM) and/or Random-Access Memory (RAM). As is well known in the art, ROM acts to transfer data and instructions uni-directionally to the processor 118, and RAM is used typically to transfer data and instructions in a bi-directional manner

Processor 118 is generally coupled to a variety of interfaces such as graphics control (e.g. graphical processing unit (GPU)), video interface, audio interface, input interface (e.g. touchscreen data input and/or keypad), and other interfaces, such as camera hardware and software components housed within the user electronic computing device for recording and transmitting, via a wireless network, digital photos, audio and video events (see FIG. 12).

Processor 118 is also coupled to a network interface that allows the processor to be wirelessly coupled to another computer (e.g. the wrap device, or telecommunications network—e.g., WiFi network, Bluetooth chip, etc.). More particularly, the network interface generally allows processor 118 to receive information from and to output information to the wireless network in the course of performing various method steps described in the embodiments herein by, for example, transferring data to and from one or more of the following: the wrap device, a user cloud storage account, a clinician's office computer storing patients' records, etc.

The user electronic computing device has installed within the device's memory 120 a unit comprising the mobile application 220 of the present invention, which may further comprise: a native application, a web application, or a widget type application to carry out the methods of the embodiments disclosed herein for receiving and transmitting treatment data and instructions to and from the wrap device. In a preferred embodiment, a mobile application 220 (e.g. a computer program product) is installed on the device 200 by downloading from the Internet. It may be written in a language to run on a variety of different types of use electronic computing devices; or it may be written in a device-specific computer programming language for a specific type of device.

In one embodiment, the mobile application 220 for communicating wirelessly with the wrap device (e.g. via Bluetooth chips) comprises: a non-transitory computer-readable storage medium storing instructions that, when executed by the processor 118, cause the user electronic computing device 200 to transmit and receive data from the wrapper device 100, by performing the steps of FIG. 11. User selections may be input into the LLLT wrap display and transmitted wirelessly to the mobile application 220 or vice versa.

Per the method of use of the wrap device 100: before treatment, the user cleans and dries the specific anatomical area (Back/Knee/Wrist/Ankle & Foot/Neck & Shoulder) to be treated so that the skin is conducive to treatment. Soap and water or alcohol is appropriate.

The user (optionally) utilizes the mobile app 220 to synchronize with the LLLT wrap device 100 and select the treatment protocol (red laser surface or deep tissue IR laser treatment) and the power setting for the session (⅓, ½, ⅔ or full). As illustrated in FIG. 11, step 1120, the user electronic computing device 200 receives the user input for activating the mobile application 220 by setting up a user account with secure login credentials, and a cloud storage option, and input further connecting the user electronic computing device 200 wirelessly with the LLLT wrap device 100 (e.g. by pairing or syncing the Bluetooth chips in the wrap with the smartphone).

The user places the LLLT wrap device 100 that is designed to fit the specific user anatomy (Small, Medium, Large) and for the specific treatment area (wraps around the user's back, knee, wrist, ankle and foot, neck and shoulder) and straps it on securely using the fixation members comprising, for example, Velcro-like tabs, straps, hooks, snaps, etc.

In step 1140, the user electronic computing device 200 and/or the LLLT wrap display (FIG. 9, 94) receives the user input for their selection of a pre-programmed, pre-calibrated treatment protocol for the particular type of wrap they are using (e.g. knee, back, etc.). In an embodiment, the mobile application 220 of the present invention is universal to all types of LLLT wrap devices, therefore, the user must select which type of wrap that they are using from a plurality of listed wrap types. In another embodiment, the mobile application does not require the user to select the type of wrap, e.g. the mobile application detects the type of wrap.

The user must also select between a surface treatment session (e.g. about 630-670 nm) penetrating the skin tissue about 1 cm depth, or a deep penetration treatment session (e.g. about 780-904 nm penetrating the skin tissue about 4-5 centimeters) on the mobile application 220, or on the LLLT wrap display 94. If the former, then the user input is wirelessly transmitted to the LLLT microprocessor 92 via the Bluetooth chip (unit 96), which activates the power switch 95 to “On”. The laser diodes 97 then emit the pre-set treatment dose (duration and intensity), and shut off automatically at the end of the treatment session.

The user also enters their pain management data into the mobile application 220 or the display 94. If the later, then the user electronic computing device 200 receives the pain management data wirelessly from the LLT wrap 100. In either case, the pain management data comprises, by way of non-limiting examples: doses of pain relief medication (over-the-counter and prescription) that the user has recently taken; and user self-assessed levels of pain.

In step 1160 the treatment session is activated, and the user electronic computing device 200 receives treatment history data (e.g. duration) and sensor data wirelessly from the LLLT wrap circuit 90, then processes and displays output on the user device GUI for the user to read before, during and after the treatment session, comprising: safety sensor 98 data (e.g. the lasers are overheating and the wrap device is shutting off); and patient sensor data 99 of the user's vital signs to ensure that they are not experiencing any adverse side effects from the treatment; and timer data (counting tip or down the minutes of each treatment session; etc.).

The LLLT wrap device emits a dose for a surface or a deep tissue treatment session comprising: between 630 nM-904 nm (nanometers) irradiation wavelength, with a power density (irradiance) between 25 mw to 500 mW, generating 1-10 joules/cm2 proven (similar to clinical LLLT products) to effectively reduce inflammation and pain, induce anti-inflammatory cellular activities, induce skin rejuvenation, and/or induce cellular level healing activities. When the dose has been delivered, the LLLT wrap device automatically shuts off. Hence, each type of LLLT wrap device is pre-programmed to operate for a specific duration (e.g. up to 20 minutes, but normally 5-12 minutes) and emit a specific amount of energy density and for the specific joint and/or medical condition for maximum pain relief. The user can pause the LLLT wrap device any time to attend to other things and resume to completion. After the treatment session ends, the LLLT wrap device 100 automatically shuts off.

In step 1180, the user data (both transmitted from the LLLT wrap device 100 and user inputted into their electronic computing device 200) is stored on the user device's memory and/or their secure cloud storage account. And if the user desires, the data (pain, sensor, treatment sessions history) may be electronically transmitted (e.g. via email) to their clinician's computers for storage in their patient file in order to receive remote supervision by their clinician on their medical treatments using the LLLT wrap device.

In one embodiment, a data table is stored in the LLLT wrap microprocessor memory and the user electronic device memory, and the data includes an identifying code for each previous treatment (for example the date), and the associated dosage(s), treatment times, codes for treatment locations, and other treatment information from previous treatments. In one embodiment, the patent data information includes a code for specifying the level and location of the patient's pain on each previous treatment, and is inputted by the patient using the screen display on the device.

Ergonomic Fit

The various types of LLLT wrap device of the present invention are shaped for encircling or covering a particular area of a user's anatomical or body part, and may comprise a variety of shapes with different types of fixation members for attaching the wrap securely to the user's body (e.g. Velcro-like straps, hooks, snaps, etc.). Each device is also a unisex wrap that comes with different sizes (S/M/L/XL) to fit all user-patients.

Each wrap device may further comprise a variety of types of materials (e.g. rubber/neoprene/cloth/resin) to make it flexible to wrap round snuggly around a user's body while providing enough stiffness to provide structural support to function also as an orthopedic brace. The wrap devices may also include other semi-stiff components (e.g. bendable plastic or metal rod members) normally found in orthopedic flexible braces (e.g. knee sport braces). Furthermore, as an example—since the patient wears ergonomic wraps—the tight fit provides additional joint pain relief similar to the arthritic gloves available in the marketplace today.

The present invention also includes the positioning of the laser diodes 97 in the exact locations required around the afflicted body area for the most effective treatment prescribed by leading orthopedic and chiropractor and laser acupuncture practicing professionals based on extensive medical research available on LLLT for pain relief today. These laser diodes are embedded into the orthopedic wraps at these exact locations such that only their lens is visible towards the treated areas.

It is also noted that the laser wrap devices are novel in the delivery method of the solution because the clinical strength lasers are integrated within the laser orthopedic wrap device in a unique combination and configuration that provides maximum pain-relief efficacy. This is due to how the ergonomic orthopedic wrap fits the user snugly for the specific size while providing an accurate delivery of the pre-determined laser energy to the required areas. This is also done while the wrap device is functioning as a brace to provide structural support to the joint to allow the joint muscles to rest. The pre-programmed wavelength provides the injured area the necessary dosage at the proper skin depth penetration, and combined with the pre-calibrated treatment times for the specific joint area, enables the LLLT wrap device to provide a highly effective treatment regime.

TABLE 1

| | |
|---|---|
| 1. Coherence | Perfect coherence (i.e. uniform dose of |
| | laser area) all LLLT wrap devices have |
| | a coherent beam over an area as large |
| | as 1.4-4.5 cm² based on the joint being |
| | treated. |
| 2. Treatment Area/Time | A very large area of 4.5 cm^{2 is} covered |
| | in each treatment area and time with |
| | one completely coherent beam. Due to |
| | this high efficiency, a smaller number |
| | of shorter treatments are required. |
| 3. Peak power | High - up to 200 mW (a half watt) in |
| | micro-pulses or regular, which |
| | guarantees maximal penetration and |
| | effectiveness. |
| 4. Energy density | High- average 0.9-20 joules per minute |
| | per cm^{2.} |
| 5. Peak energy/minute | 14.4 joules over the entire treated area. |
| | Extremely high power contributes to |
| | greater effectiveness. |
| 6. Micro-pulses | Unlike continuous waves, micro-pulses |
| | allow the beam's power and ability to |
| | deeply penetrate to the source of the |
| | problem, thus increasing the treatment's |
| | effectiveness. |
| 7. Weight | 150-200 g - light, portable and |
| | rechargeable - ready to use anywhere at |
| | any given moment. |
| 8. Operation methods | Wear it and turn device on by pressing |
| | the ON button. Device automatically |
| | shuts-off after predetermined treatments. |
| | Counter displays usage time and No. of |
| | usages and other metrics. |
| 9. Historic Pain Data | User can capture pain measurement data |
| | on devices. Pain data auto-synchs via |
| | WiFi, or Bluetooth ® with Smartphone |
| | application provided with device. |
| 10 Sensors | A variety of sensors are utilized in the |
| | device: |
| | Safety Sensors - Temp sensors and |
| | proximity sensors for patient safety |
| | Vitals sign monitoring sensors - |
| | Monitoring sensors patient vitals such |
| | as Body Temperature. Blood Pressure |
| 11. Safety googles | No need for safety goggles - Class 1 |
| | safety approval. |
| 12. Wrap Types | Laser Wrap LW-Back-1000 Sample |
| | specifications (For Back joint related |
| | Pain relief) |
| | Laser Wrap LW-Knee-1000 Sample |
| | specifications (For Knee joint related |
| | Pain relief) |
| | Laser Wrap LW-Wrist-1000 Sample |
| | specifications (For Wrist joints related |
| | Pain relief) |
| | Laser Wrap LW-Ankle & Foot-1000 |
| | Sample specifications (For Ankle & |
| | Foot joints related Pain relief) |
| | Laser Wrap LW-Neck & Shoulder-1000 |
| | Sample specifications (For Neck & |
| | Shoulder joints related Pain relief) |
| | Laser Wrap LW-Elbow-1000 Sample |
| | specifications (For Elbow joints related |
| | Pain relief) |

According to one embodiment, the present invention provides a complete system of plurality of wearable devices and an artificial intelligence or machine learning powered smart platform with ‘smart virtual assistant’ for monitoring and treatment of the peripheral neuropathy caused by the high blood sugar, muscle pain, osteoporosis, arthritis, Carpel Tunnel, Tennis Elbow, Plantar Fasciitis, and other joint illnesses different wrap devices and the computer implemented AI/ML powered platform.

As disclosed above, the plurality of wearable devices are LLLT wrap devices for different parts of the body such as arm, leg, ankle-foot, knee, back, neck-shoulder etc. for treatment of pain within said part of the user's body using the laser therapy. According to one embodiment, the wearable devices are devices which wraps around its respective part of the body such that the plurality of sets of laser diodes embedded within the wearable device contacts the skin of the body to emit the laser light of infrared or near infrared wavelength. A plurality of various sensors are further embedded within the plurality of LLLT wrap device to monitor and capture the vital data of the user to monitor and treat pain being caused by peripheral neuropathy, osteoporosis, arthritis, Carpel Tunnel, Tennis Elbow, Plantar Fasciitis, or any other joint illnesses.

The system according to present invention includes sets of laser emission diodes of different wavelength embedded within the wearable devices for treatment of different level such as skin treatment or deep penetration, depending on the cause of pain. All the LLLT wearable devices of present invention is comprising of a plurality of body monitoring sensors specially continuous glucose monitoring sensors, body dietary/nutrition markers (Ketone levels, Insulin levels, etc.) & related data and motion detection sensor to detect movement of respective part to which the device is wrapped. Both these sensors are configured within each LLLT wrap device to monitor the condition of peripheral neuropathy caused due to the high level of blood sugar or diabetes. According to present embodiment, as explained earlier, the plurality of LLLT wrap devices further includes other vital sensors for monitoring other vital data of the user such as temperature, blood pressure. EKG, heart rate, respiratory rate, etc. The LLLT wrap devices of present system further includes a temperature sensors to detect the temperature of the laser diodes and the temperature of near skin on which the LLLT wrap device is wrapped to avoid burning of skin of the user.

According to one embodiment, the plurality of LLLT devices of present system further includes an electronic unit having a computer processor to collect, decode and transmit the real time sensory data to the centralized cloud server via a communication module. The centralized cloud server of present system is coupled with the AI powered computer implemented platform accessible by any user, care givers, medical personnel, loved ones of family of the patient or by any other person of concern using his/her smart computing device such as mobile phones, computer, laptops, smart watch and/or any other wearable smart device.

The AI and/or ML powered computer implemented platform with a smart virtual assistant of present system includes an artificial intelligence and a machine learning algorithms stored within the platform that accesses real time vital sensor data from the cloud server, processes said data and accordingly manipulates working operation of the plurality of the LLLT wrap devices. According to one embodiment, the computer implemented platform further notifies the personal physician or concern medical person in case of detection of any medical condition or in case of detection of any vital body data above or below the normal range of said vital body data.

According to one embodiment, the AI and/or ML powered computer implemented platform with a PSA is further capable of being interfaced with any other third party medical devices or platforms thus capable of being accessed through such third party medical devices, for example, via Fitbit, Google Health, Amazon-Alexa etc. The AI powered computer implemented platform of present system according to one embodiment, acquires real time data from the centralized cloud server, process such data to determine any emergency conditions and accordingly alerts the user and all the associated concern persons of the user via the computer implemented platform installed within the smart computing devices of them.

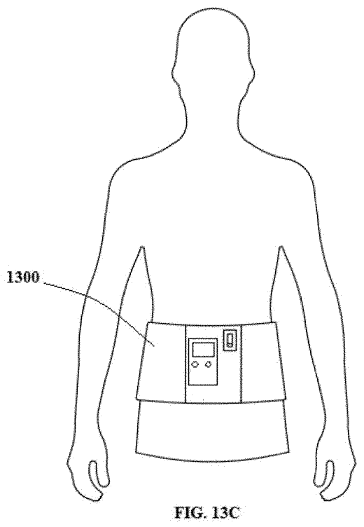

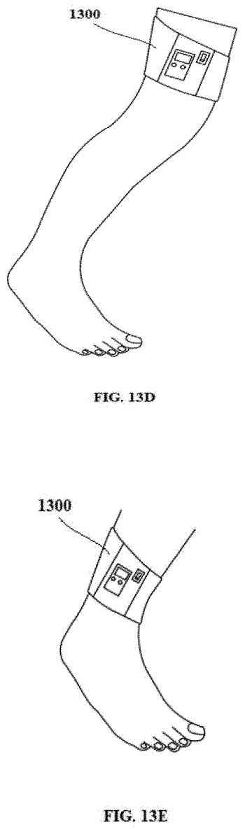

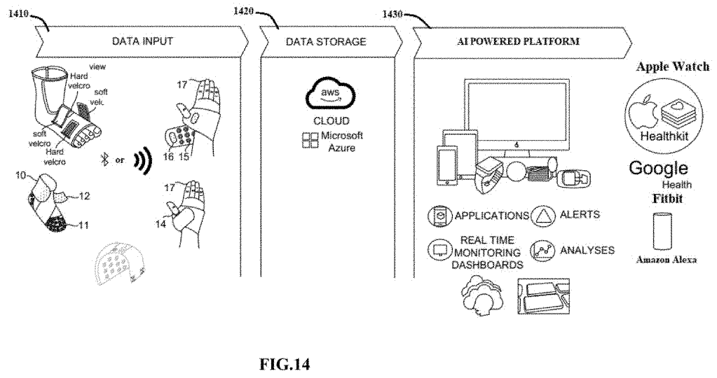

FIG. 13A shows flat view of one another embodiment of the flexible wrap 1300 (well wrap flex) of present invention. According to present embodiment, the flexible wrap is capable of being wrapped around any part of the body without consideration of said part of the body, whether it's a muscle curvature or the joint specific curvature. The material used for this embodiment is flexible plastic which can be molded onto the body part as needed for a comfortable fit, and ensure optimal treatments for the said body part. According to present embodiment, the wrap 1300 is comprising of flexible main body 1310 with a plurality of low level lased emitting diodes 1320 embedded within it and an electronic circuit unit 1330 configured at the center having all the electronic components of the wrap device 1300. FIG. 13B shows a turned view of the same flexible wrap 1300, that may easily be wrapped around the curvature of the body as needed. According to present embodiment, the electronic unit 1330 of present flexible wrap device 1300 further includes a touch sensible display 1340 to allow user to see the sensory data over the display as well as to control the device 1300 using the touch sensible screen 1340, a plurality of various sensors 1360 for monitoring various body data such as blood sugar level, body flexibility, respiration rate as well as body skin monitoring sensor and wrap temperature monitoring sensor to avoid extra heating and burning of skin due to overheating from laser diodes 1320.

According to one embodiment, the electronic unit 1330 further includes an in-built rechargeable battery 1370 for working of the electronic components of the wrap 1300. A processor and a communication module are also configured within the electronic unit 1330 to allow the wrap 1300 to transmit the real time data over the computer implemented platform of the system as well as to store said data over the centralize server unit. According to one embodiment, the wrap 1300 further includes a manual switch 1350 to manually turn ON or OFF the wrap device by the user, when needed. According to present embodiment, the system further includes additional health-vital sensors embedded within the wrap 1300 at different location for accurate reading by the sensors. Such health-vital sensors includes temperature monitoring sensors, blood pressure monitoring sensors. EKG etc. FIGS. 13C, 13D and 13E shows a flexible wrap 1300 of present system wrapped around different parts of the body to explain that the present wrap is capable of being wrapped around or conform any part of the body to treat the pain. The present embodiment of the wrap allows user to use the wrap at any part of the body without limiting its user mere at the back.

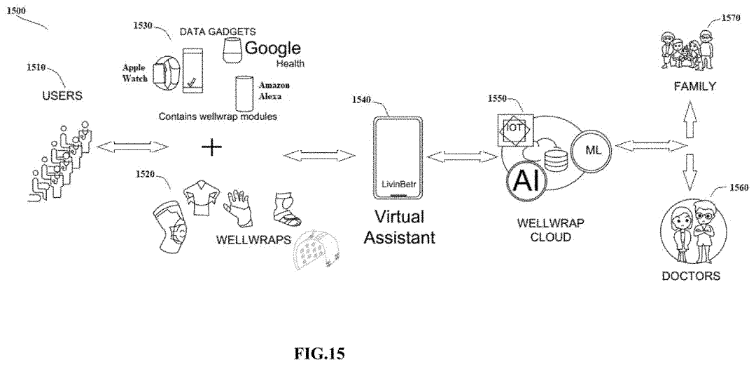

Referring to FIG. 14 now which shows a data flow diagram of present pain monitoring and treatment system wherein the system is made of plurality of LLLT wrap devices as a data input units 1410 that are capable of monitoring as well as treating the parts of the body with pain using the low level laser therapy. All the LLLT wrap devices of the system includes a plurality of laser diodes to emit the laser light of uniform wavelength and intensity at the respective part of the body of user for specific amount of time pre-stored within the processor of the device which is further manipulated by the AI MI-PSA powered computer implemented platform depending on the real time condition of the user.

The LLLT devices of the system works as a data input unit which includes plurality of different vital data sensors to monitor the vital body data of the user in real time. Each LLLT device of present invention further includes an electronic circuit or unit having a processor and a communication module to transmit the data from sensors to the cloud server of the system which works as a data storage unit 1420 of present system. According to one embodiment, the data storage unit or a cloud server 1420 is a centralized server to store the data from the data inputs 1410 in real time. The centralized cloud server of present invention is any third party cloud server.

According to one embodiment, the present cloud server 1420 is capable of being accessed by the AI powered computer implemented platform with PSA 1430 for data acquisition, data interrogation and manipulation. The computer implemented platform acquires said real time sensory data from the cloud server 1420, processes said data using machine learning algorithms and artificial intelligence of present computer implemented platform 1430 and provides real time dashboard of said data over the platform for the user and the person of concern to the user.

According to one embodiment, the computer implemented platform, based on analysis of real time vital sensor data using the AI and machine learning algorithms, alerts the user as well as medical persons of concern and family members, in case any situation arises such as increase of any vital body data from a threshold normal limit. This threshold data limit is set by the system for doctor approval and/or by the doctor or personal physician for the said patient. Each patient's specific questions are tailored to the existing conditions that the patient currently has and the questions are also approved by the patient's doctor such that its relevant personalized medicine, may be provided for better ‘personalized’ patient outcomes. The computer implemented platform with smart virtual assistant of present invention alerts the user and all the concern persons by vibrating the smart device of the user or via sound, text or message notification over the smart device of the user. The computer implemented platform 1430 of present invention is further capable of being interfaced with the third party healthcare devices or platforms such as Amazon Alexa, Google fitbit, and Apple Health—watch etc.

According to one embodiment, the computer implemented platform 1430 with PSA of present invention further behaves as a smart assistant for the patient or user, where the platform 1430 analyzes vital sensory data of the user continuously, and based on pre-determined set conditions, launches a screen interactive questionnaire for the user in which the user is asked to answer specific questions that generally a personal physician would ask when interviewing the user for the first time. The AI and the machine learning algorithms, based on the analyzed vital body data as well as past medical history of the user, prepares a questionnaire for the user.

This questionnaire is originally available in the cloud server and the AI and/or ML powered platform of present invention chooses the standard care questions that makes sense for the specific patient, which is also personalized to the said patients existing conditions, and make it available for the patient and his physician to approve and to use. According to one embodiment, the prepared questions by the AI/ML powered smart virtual assistant of the platform are further edited and finalized and confirmed by the personal physician such that it's uniquely tailored to the specific patient with the triggers set by the physician to enable these Q&A again specific to the user (For example—if the patient is diabetic and know for high blood pressure—the doctor sets the trigger as 3 continues days of 90/160 where the system triggers the Q&A and get the answers quickly to the physician so point of care with immediate intervention can lead to better patient outcomes.

According to one embodiment, the laser wrap platform is also capable to execute voice enabled Q&A session via system integrated third party platforms of choice of user, such as, via Apple Siri on the phone, or via Amazon Alexa or using Google home assistant. The computer implemented platform 1430 with PSA of present invention allows user to answer said question by many different ways using the smart user device in which the present computer implemented platform is installed or by using any other synced third party healthcare device with the platform 1430 interfaced. The system allows user to submit the answers of the questions raised by the platform 1430 via text or voice on the smart phone with the platform installed or through any other interfaced third party devices such as Amazon Alexa, Apple Siri, Google Assistant etc.

FIG. 15 shows a flow diagram of present system 1500 with an integrated virtual assistant within the platform. According to present embodiment, the system 1500 includes an AI and/or ML backed virtual assistant that depending on the preset triggers by the personal physician of the patient, creates a customized ‘personalized’ questions based on the real time vital sensory data of the patient, which the physician may normally ask the patient before consultation. According to present embodiment, the virtual assistant 1540 of present computer implemented platform is in continuously contact with the well wrap devices 1520 and the third party healthcare devices 1530 of the user 1510 integrated with the present system 1500.

Further, according to present embodiment, the virtual assistant 1540 of the system 1500 further stores said data and questionnaire over the cloud server 1550 for the personal physician 1560 and the family members or loved ones 1570 to access it through their personal smart devices. According to one embodiment, the well wrap devices is any wrap device of present system 1500 wrapped around any body part of the user 1510. According to one embodiment, the third party data gadgets 1530 integrated with present system 1500 is any of the healthcare or smart device such as smart wearable devices (smart watches, bands etc.) or any other healthcare device such as Amazon Alexa, Google Health, etc.

### CONCLUSION

The present invention comprises a flexible home treatment with AI/ML data insights, that enables self-care and immediate treatments, thus improving patient lives while providing for maximum pain relief for the specific related illnesses like peripheral neurapathy, osteoporosis and arthritis, Carpel Tunnel, Tennis Elbow, Plantar Fasciitis, and other joint illnesses.

The present invention's wear-ability provides portable and hands-free convenience using laser wraps-orthopedic braces for the affected areas, with accurate proximity and automated timers for optimal treatment for joint pain relief, and accelerated healing.

The consumer buys it once and treats himself/herself conveniently and easily by wearing the device, and simply pressing the ON/OFF button, at the comfort of their home or anywhere while going about their daily activities.

The present invention, since its battery operated—is highly portable for use anywhere patient decides, when they actually feel the pain, making it extremely efficient and convenient, and not having to take invasive drugs, while also avoiding the side effects. It comes with a convenient device holder and DC charger.

The laser wrap devices of the present invention are already preset using the mobile application controlled by the user, for the optimum clinical strength energy density (i.e. dose) required for the specific area, so the user just has to use the ON/OFF switch for treatment, making it the most convenient device for patient use in the industry.

Although various features of the invention may be described in the context of a single embodiment, the features may also be provided separately or in any suitable combination. Conversely, although the invention may be described herein in the context of separate embodiments for clarity, the invention may also be implemented in a single embodiment.

As used herein, the term “about” refers to plus or minus 5 units (e.g. percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

1. A portable low level laser therapy (LLLT) wrap system for treatment of pain from peripheral neuropathy, comprising:
a plurality of flexible wrap devices with different embodiments for different parts of a body of a user;
a computer implemented platform with an Artificial intelligence or Machine learning powered virtual assistant; and
a central cloud server;
wherein each of the plurality of flexible wrap device is further comprising:
a flexible main body of a wrap;
one or more fixation members to secure the flexible main body of the wrap to the user's body, comprising Velcro-like tabs, straps, buckles, ties, hooks or snaps;
a plurality of laser diodes embedded within the flexible main body of the wrap to emit low level laser light for treatment of pain;
a microprocessor;
a plurality of sensors able to monitor the wrap device and a body vital data of the user; and
a wireless data transceiver unit for wirelessly transmitting and storing a treatment data and a sensory data over the central cloud server.

2. The portable low level laser therapy (LLLT) wrap system of claim 1, wherein the medical conditions comprise pain associated with: peripheral neuropathy caused due to high level of blood sugar or other neurological diseases.

3. The portable low level laser therapy (LLLT) wrap system of claim 1, wherein the medical conditions comprise pain associated with: peripheral neuropathy and tendonitis of the back, knee, hand, and Achilles tendon; tennis elbow; carpel-tunnel: arthritis (rheumatoid and osteoarthritis); osteoporosis; plantar fasciitis; bursitis; muscle and/or tissue inflammation and damage from acute and chronic injuries.

4. The portable low level laser therapy (LLLT) wrap system of claim 1, wherein the plurality of flexible wrap devices with different embodiments for different parts of the includes at least one of: a back; a knee; an ankle-foot; a hand-wrist; a neck-shoulder; and an elbow device.

5. The portable low level laser therapy (LLLT) wrap system of claim 1, wherein plurality of laser diodes includes two different types of laser diodes to emit different doses for either a skin surface treatment, or a deep surface treatment, during a treatment session.

6. The portable low level laser therapy (LLLT) wrap system of claim 1, wherein the plurality of sensors includes continuous glucose monitoring sensor (CGM) to monitor blood glucose level, motion sensor to continuously monitor flexibility of the user, body dietary and nutrition markers such as keton levels, insulin levels, and other vital sensor to monitor temperature, blood pressure and EKG.

7. The portable low level laser therapy (LLLT) wrap system of claim 1, wherein the computer implemented platform with an Artificial intelligence or Machine learning powered virtual assistant is acquires treatment data and sensory data from the central cloud server, processes using an AI or ML and notifies a personal physician or family members of the user, in case of any medical emergency detected.

8. The portable low level laser therapy (LLLT) wrap system of claim 1, wherein the Artificial intelligence or Machine learning powered virtual assistant prepares a customized user specific questionnaire for the user based on the predefined trigger by the personal physician or based on the real time sensory data.

9. The portable low level laser therapy (LLLT) wrap system of claim 1, wherein the Artificial intelligence or Machine learning powered virtual assistant provides said questionnaire to the user and also stores over the central cloud server.

10. The portable low level laser therapy (LLLT) wrap system of claim 1, wherein the questionnaire includes specific ‘personalized’ questions which the personal physician may ask based on health condition of the user when consulted.

11. The portable low level laser therapy (LLLT) wrap system of claim 1, wherein the computer implemented platform further integrates third party health devices though which the user may answer the questions of the questionnaire.

12. The portable low level laser therapy (LLLT) wrap system of claim 1, wherein the user may answer the questionnaire through mobile message, email, mobile audio or through any other integrated third party medical health devices.

13. The portable low level laser therapy (LLLT) wrap system of claim 1, wherein the computer implemented platform transmits the answer of the user to the personal physician over his or her personal smart device and also stores the answer of the user over the central cloud server.
